(19) United States
(12) Plant Patent Application Publication
(10) Pub. No.: US 2008/0184412 P1
(43) Pub. Date: Jul. 31, 2008

(54) Grevillea plant named 'Callum's Gold'

(76) Inventor: Brad Nielsen, Queensland (AU)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/699,928

(22) Filed: Jan. 30, 2007

(51) Int. Cl. A01H 5/00

(52) U.S. Cl. PLT/226

(57) ABSTRACT

A new and distinct variety of Grevillea, named Grevillea sp. ‘Callum's Gold’, is provided. This plant is characterized by its flower color, dark yellow, and it's compact, dense foliage.

### LATIN NAME

Grevillea sp.

### VARIETY DENOMINATION

‘Callum's Gold’

### BACKGROUND OF THE INVENTION

The present invention relates to a new and distinct Grevillea sp. cultivar known as ‘Callum's Gold’. Grevillea are evergreen flowering shrubs or trees that are endemic to Australia and are grown for their beautiful fern-like foliage and colorful inflorescences of small tubular flowers. They are generally grown outside in mild climate areas of the United States, such as California. The inflorescences comprise flowers, typically in reds, yellows, or pinks, that are about half of an inch long with long styles. While most Grevillea sp. grow as low prostrate shrubs, many species can become large trees.

The Grevillea sp. are members of the Family Proteaceae. The unique structure of the flower organs, of many individuals of this Family make it difficult for normal pollination to occur. Thus, to ensure pollination, the anthers of the flowers, prior to anthesis, will dehisce and deposit their pollen onto the tip of the style. After anthesis, the style protrudes prominantly from the flower allowing distribution of the pollen. The tip of the protruding style is referred to as a “pollen presenter” because it, rather than the anthers, distributes pollen.

### BRIEF SUMMARY OF INVENTION

A new and distinct cultivar of Grevillea sp., variety designation ‘Callum's Gold’, is provided. The Grevillea cultivar of the subject invention is characterized by its unique very dark yellow, tropical brush flowers and short, compact, bushy foliage. The subject cultivar produces an attractive bush-like tree with eye-catching flowers and is suitable for potting or landscaping uses in a variety of gardens.

The new subject cultivar arose as a spontaneous seedling among open-pollinated stock of the Grevillea cultivar “Honey Gem” (not patented) in a nursery in Beenleigh, Queensland, Australia. The suspected seed parent, ‘Honey Gem’, produces inflorescences whose flowers have characteristic apricot colored parenaths and orange styles and open, moderately dense foliage growing up to 6 meters tall. Upon observation of development, it was seen that the new cultivar “Callum's Gold” had distinctly dark yellow flowers and dark green, deeply-lobed, more compact, dense foliage and was self-pollinating. The cultivar was selected for future propagation because of these desirable observed characteristics.

A possible male parent may be the Grevillea var. “Sandra Gordon” (not patented), which was in close proximity to the “Honey Gem” stock. This cultivar is characterized with shiny green, pinnately lobed leaves growing as an open, sprawling plant approximately 6 meters tall, with the flowers of the inflorescences having grey-green perianths and yellow styles.

The subject plant cultivare was asexually reproduced from cutting material. Observation of asexually produced plants of three successive generations showed that the desirable characteristics were stable and reproduced true to type within each generation.

This plant was selected because of its advantageous characteristics of compact, bushy growth habit and self-pollinating, very dark yellow flowers that are distinctively different from other known cultivares of Grevillea sp. The closest comparative Grevillea cultivare is “Yamba Sunshine”, but there are distinct differences between the foliage and flower color of these cultivares. Table I presents general characteristics that are distinctive for Grevillea sp. cultivares “Callum's Gold”, “Honey Gem”, and “Yamba Sunshine”. These distinctive characteristics were observed by a breeder maintaining the asexually reproduced candidate cultivare, as well as the comparative cultivares, in Burpengarry, Queensland, Australia.

TABLE I

| | Grevillea sp. cultivares observed | | |
|---|---|---|---|
| | ‘Callum's | ‘Honey | ‘Yamba |
| Characteristic Observed | Gold’ | Gem’ | Sunshine’ |
| Young stem color | Greyed orange | Brown | Greyed orange |
| Stem color | Greyed purple | Brown | Brown |
| Petiole length | Medium | Medium | Long |
| Leaf attitude to stem | Semi-erect | Semi-erect | Horozontal |
| Leaf color, lower side | White | White | Light green |
| including hairs | | | |
| Leaf degree of hairiness | Medium | Weak | Weak |
| on upper side | | | |
| Leaf Shape of apex of | Flattened | Flattened | Pointed |
| sinus | | | |
| Inflorescence length | Medium | Long | Medium |
| Inflorescence predominant | Yellow | Orange | Yellow |
| color | | | |
| Inflorescence attitude | Horizontal | Semi-erect | Horizontal |
| Bud: color of limb | Green | Yellow | Yellow |
| Perianth color | Yellow | Orange | Yellow |
| Nectary color | Orange | Orange | Yellow |
| Curvature of Style (after | Straight | Straight | Gently curved |
| anthesis, before | | | |
| dehiscence of perianth) | | | |

An application for the grant of Australian Plant Breeders Rights for ‘Callum's Gold’ has been filed as application number 2005/182. The application was accepted for publication on Jun. 29, 2005, and is currently pending in the Plant Breeders Rights Office of Australia.

### BRIEF DESCRIPTION OF THE DRAWINGS

FIG. 1 Photograph of a typical field specimen of Grevillea sp. variety ‘Callum's Gold’

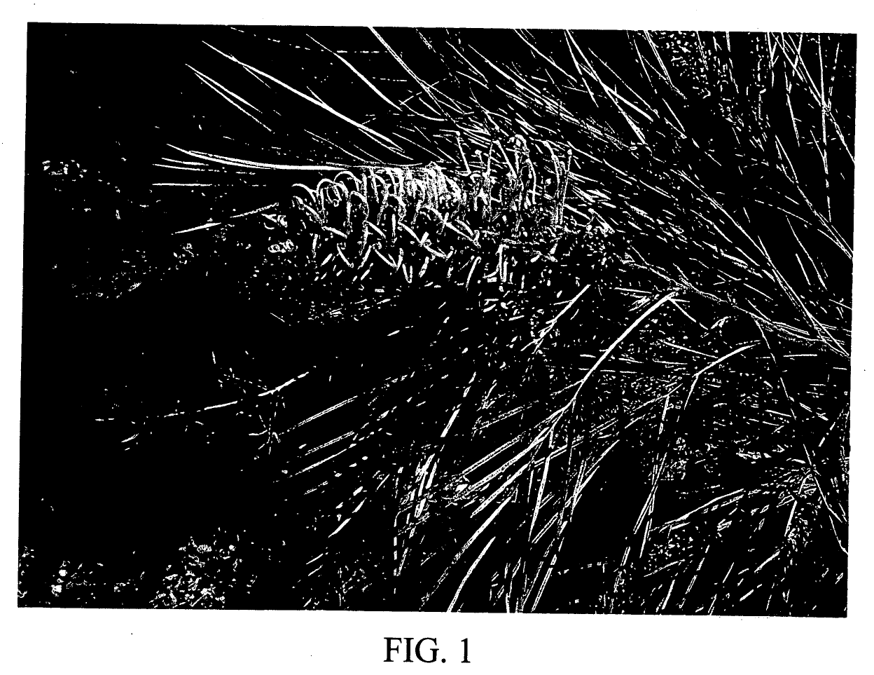

FIG. 2 Photograph comparing inflorescences of three different varieties of Grevillea sp.; ‘Callum's Gold’, ‘Honey Gem’, and ‘Sandra Gordon’.

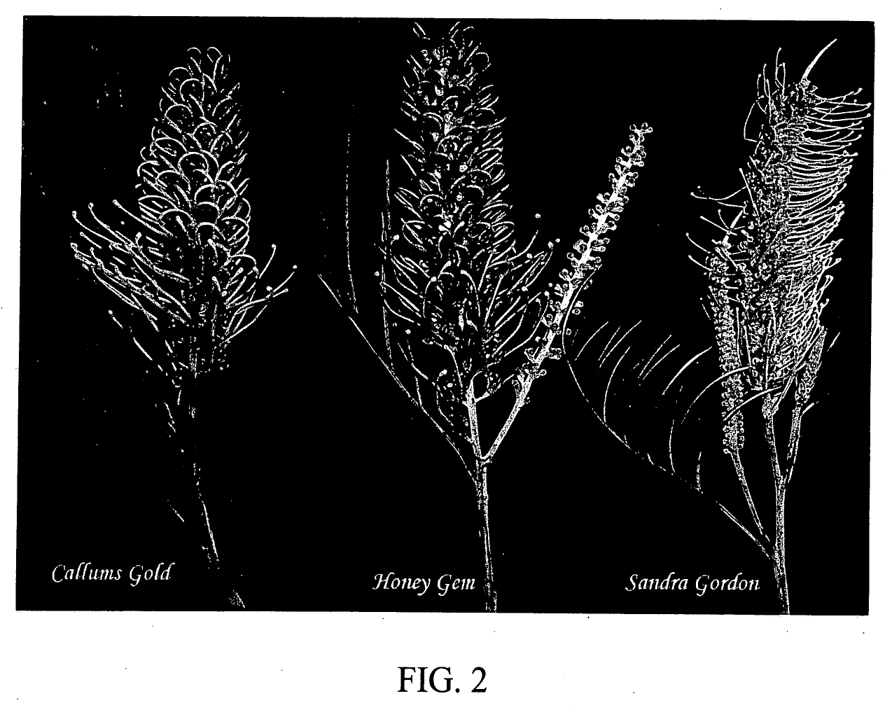

FIG. 2 Photograph comparing typical leaves of three different varieties of Grevillea sp.; ‘Callum's Gold’, ‘Honey Gem’ and, ‘Sandra Gordon’.

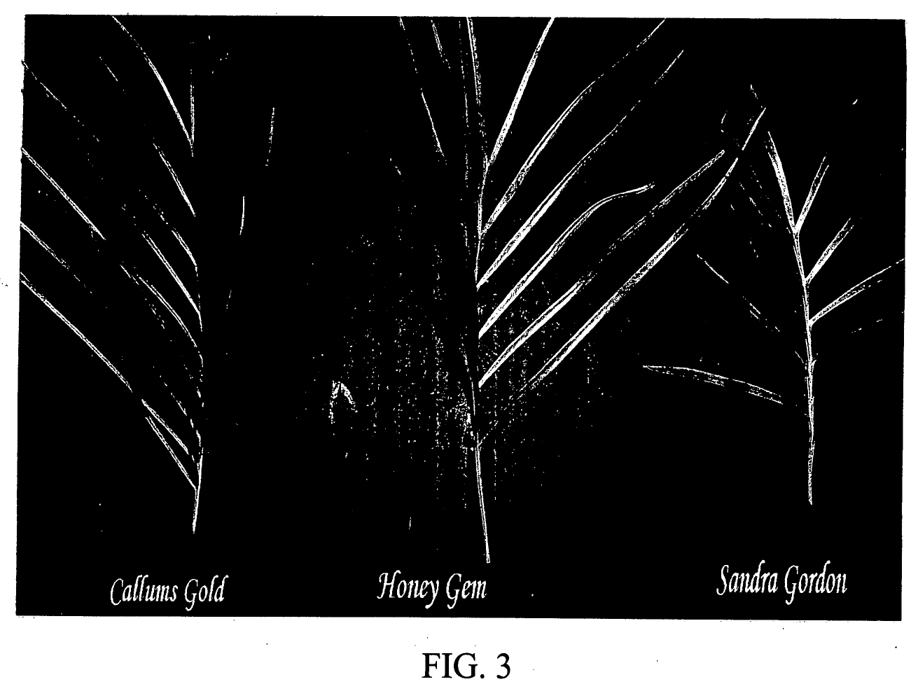

FIG. 4 Photograph of Grevillea sp. ‘Yamba Sunshine’ inflorescence. This variety is similar to the variety ‘Callum's Gold’ of the subject application, but has distinct observable differences in the foliage as well as paler flowers.

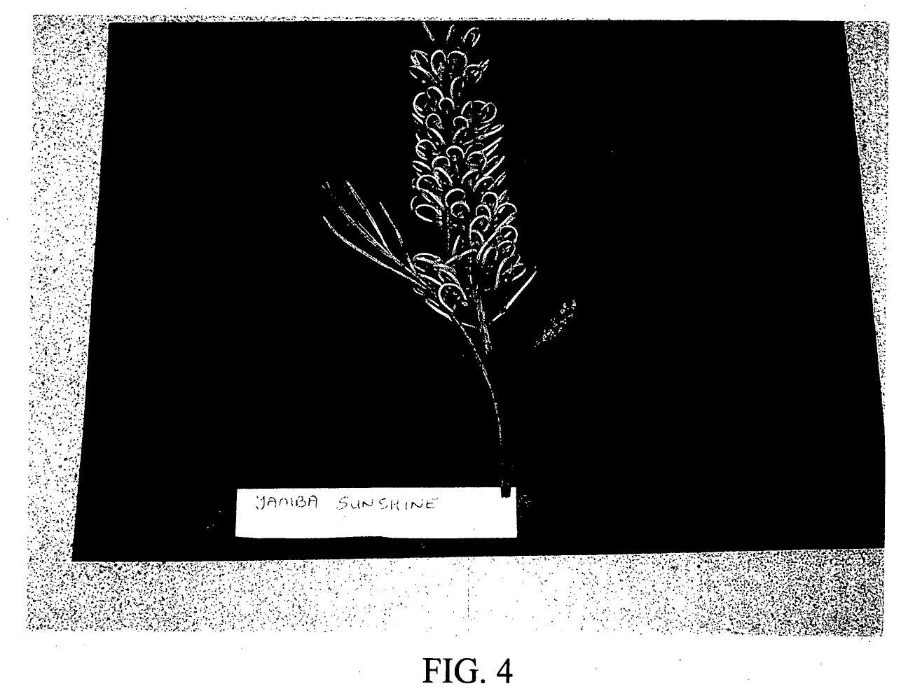

FIG. 5 Photograph of typical leaves of Grevillea sp. ‘Yamba Sunshine’.

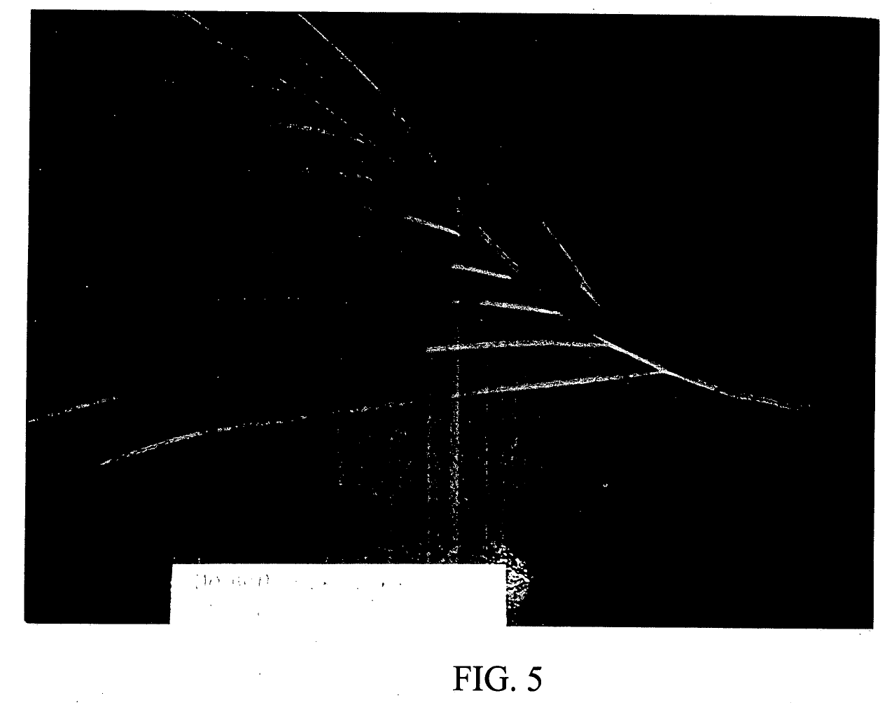

### DETAILED BOTANICAL DESCRIPTION

The following observations, measurements and comparisons were taken from ten (10) individual tube stocks of the cultivar grown in Burpengary, Queensland, Australia. Cultivars were planted in 200 mm pots of standard bark potting mix and placed in an open sun area.

The following description uses color references to The Royal Horticultural Society (R.H.S.) Color Chart, 1986 edition, except where general terms of ordinary dictionary significance are used.
Botanical classification: Grevillea sp. hybrid cultivar “Callum's Gold”
Original material: Seedling
Scientific name: Grevillea sp.
Varietal name: ‘Callum's Gold’
Parentage:
   Female parent.—likely Grevillea sp. “Honey Gem” (G. banksii×pteridifolia).
   Male parent.—possibly Grevillea sp. “Sandra Gordon” (G. sessilis×pteridifolia), which was in close proximity.
   Propagation.—Grevillea sp. ‘Callum's Gold’ should be reproduced from cuttings to maintain the unique characteristics.
General plant description:
   General appearance.—compact, dense shrub.
   Growth habit.—upright Attitude of Branches — erect Height — medium, between 1 to 3 meters Density of foliage at flowering: medium.
   Leaves—(general description).—some or all of the leaves are divided into lobes, with a degree of division to the third order, having sinuses greater than two thirds of the way to the midrib, with a medium number of lobes of regular spacing Margins: weak undulation; smoothly recurved, undersurface on either side of the midvein partly exposed Overall Length: very long (>20 cm) Width at widest point: broad (15-20 cm) Attitude to stem: semi-erect Attitude of longitudinal lobe axis to longitudinal midrib axis: semi-erect Attitude of longitudinal lobe axis to another on same side of leaf: parallel Shape of apex of sinus: broad, flattened Lobe length: long Lobe width: narrow Shape of Apex of ultimate lobe: pointed Color: Upper surface (including hairs): Dark green Lower surface (including hairs): White Hairiness on lower side: white Hairiness: Upper surface: medium Lower surface: long Petiole length: medium.
   Stems.—Stem color: greyed purple Young stem color: greyed orange Hairiness: strong.
   Inflorescence.—Position: both terminal and axillary Length: medium Width: medium Predominant color: yellow Density of florets: dense Number of flowers: many to very many Attitude: horizontal Form: cylindrical Branching: medium Sequence of opening of flowers: centripedal Rachis length: medium.
   Flowers.—Attitude of Pedicel: pedicels of flowers lean away from the inflorescence peduncle Length of Pedicel: short Perianth: Color: yellow Hairiness, including stem: strong Color of hairs: red brown Length: medium Width: narrow Length/width ratio: medium Coherence of tepals on dorsal side: less than ⅓ Coherence of tepals on ventral side: greater than ⅔ Tepal flanging at margin: absent or very weak Nectary: Color: orange Pistil: Length: long Length relative to perianth: much longer Stigma color: yellow Style: Color: orange Curvature (post anthesis, but prior to dehiscence of perianth): Straight Hairiness: absent or very weak Pollen Presenter attitude to style: oblique Color of Pollen Presenter: yellow Concurrence of pollen presenter with style: absent Shape of Pollen presenter: dome Pollen color: yellow Ovary: Color: green Hairiness: strong.

We claim:

1. A new and distinct variety of plant, Grevillea sp. “Callum's Gold”, as Illustrated and described herein.
